(19) United States
(12) Patent Application Publication
(10) Pub. No.: US 2011/0184060 A1
(43) Pub. Date: Jul. 28, 2011

(54) ORAL DOSAGE FORMS HAVING A HIGH LOADING OF A TRANEXAMIC ACID PRODRUG

(75) Inventors: Sarina Grace HARRIS MA, Santa Clara, CA (US); Nikhil Pargaonkar, Sunnyvale, CA (US); Sami Karaborni, Cupertino, CA (US)

(73) Assignee: XenoPort, Inc.

(21) Appl. No.: 13/010,269

(22) Filed: Jan. 20, 2011

Related U.S. Application Data
(60) Provisional application No. 61/297,589, filed on Jan. 22, 2010.

(51) Int. Cl. A61K 31/27; C07C 271/22; A61P 7/04; A61P 17/02

(52) U.S. Cl. 514/506; 560/115

(57) ABSTRACT

Oral dosage forms with a high loading of 4-({[(2-methylpropanoyloxy)ethoxy]carbonylamino}methyl)cyclohexanecarboxylic acid are disclosed.

This application claims benefit of U.S. Provisional Application No. 61/297,589, filed on Jan. 22, 2010, which is incorporated by reference herein.

The present disclosure relates to sustained release oral dosage forms with a high loading of 4-({[(2-methylpropanoyloxy)ethoxy]carbonylamino}methyl)cyclohexanecarboxylic acid.

Tranexamic acid, trans-4-(aminomethyl)-cyclohexanecarboxylic acid:

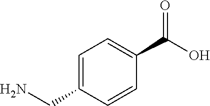

is an antifibrinolytic agent that reversibly blocks lysine binding sites on plasminogen and plasmin, and acts to prevent proteolytic degradation of fibrin clots which form in the normal physiologic process of hemostasis. Both plasminogen and plasmin are activators of fibrinolysis and active clot-lysing agents. Tranexamic acid thus helps to stabilize fibrin clots, which in turn maintains coagulation and helps to control bleeding. Tranexamic acid is used clinically to control bleeding, for example, bleeding associated with surgery, gastrointestinal hemorrhage, blood loss in subjects with advanced cancer (both acute hemorrhagic events and low-volume chronic bleeding), bleeding that occurs during dental procedures, and for heavy bleeding during menstruation (menorrhagia) (Wellington and Wagstaff, Drugs, 2003, 63, 1417-1433; and Dunn and Goa, Drugs, 1999, 57, 1005-1032; and Pereira and Phan, The Oncologist, 2004, 9, 561-570). In prophylactic uses it is desirable to administer tranexamic acid prior to bleeding and is most conveniently accomplished via oral administration. Due to the suboptimal pharmacokinetic properties of tranexamic acid, which include modest oral bioavailability (about 30%) and a rapid terminal elimination half life of about 2 hours, oral formulations such as Cyklokapron® are typically dosed at high concentrations. To address the incomplete gastrointestinal absorption of tranexamic acid, prodrug derivatives have been developed (Svahn et al., J Med Chem 1986, 29, 448-453; Svahn et al., EP 0 079 872 B1; Svahn et al., U.S. Pat. No. 4,483,867; Jonsson, WO 94/15904; Svahn et al., Arzneim-Forsch. 1988, 38, 735-738; and Edlund et al., Br J Obstet Gynaecol 1995, 102, 913-917). The prodrug 1-(ethoxycarbonyl)oxyethyl trans-4-(aminomethyl)-cyclohexane carboxylate (Kabi 2161) showed improved oral bioavailability of tranexamic acid in human subjects.

Recently, Zerangue et al. described acyloxyalkyl carbamate prodrugs of tranexamic acid that are absorbed from the large intestine and which are appropriate for oral administration using sustained release dosage forms (Zerangue et al., U.S. Pat. Nos. 7,351,740 and 7,592,369. The high oral bioavailability of these acyloxyalkyl carbamate tranexamic acid prodrugs can lead to improved convenience, efficacy, and side effect profile of tranexamic acid therapy. In particular, U.S. Pat. No. 7,351,740 discloses the tranexamic acid prodrug 4-({[(2-methylpropanoyloxy)ethoxy]carbonylamino}methyl)cyclohexanecarboxylic acid (1):

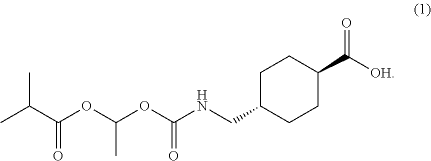

Oral dosage forms comprising compound (1) are generally disclosed in U.S. Pat. No. 7,351,740 and U.S. application Ser. No. 12/858,401 filed Aug. 17, 2010. However, the tablet formulations disclosed in these references have a loading of compound (1) that may result in large tablets to support a high drug dose, and the properties of the granulation used to prepare the tablets are not ideally suited for commercial tableting operations. Tablets comprising a high drug loading of other compounds are disclosed in U.S. Publication Nos. 2010/0226981 and 2010/0099907. The use of acyloxyalkyl carbamate prodrugs of tranexamic acid to reduce or minimize bleeding such as perioperative bleeding and in bleeding due to traumatic injury is disclosed in U.S. application Ser. No. 12/858,401 filed Aug. 17, 2010.

Thus, oral tablet dosage forms comprising a high loading of 4-({[(2-methylpropanoyloxy)ethoxy]carbonylamino}methyl)cyclohexanecarboxylic acid and that are amenable to high throughput commercial tableting manufacture are desirable.

Oral tablet dosage forms having a high loading of 4-({[(2-methylpropanoyloxy)ethoxy]carbonylamino}methyl)cyclohexanecarboxylic acid prepared from granulations with greater than at least about 80 wt-% 4-({[(2-methylpropanoyloxy)ethoxy]carbonylamino}methyl)cyclohexanecarboxylic acid are disclosed.

In a first aspect, oral tablet dosage forms are disclosed comprising about 80 wt-% to about 90 wt-% 4-({[(2-methylpropanoyloxy)ethoxy]carbonylamino}methyl)cyclohexanecarboxylic acid.

In a second aspect, solid granulations comprising greater than about 95 wt-% 4-({[(2-methylpropanoyloxy)ethoxy]carbonylamino}methyl)cyclohexanecarboxylic acid are disclosed.

In a third aspect, oral tablet dosage forms comprising a solid granulation wherein the granules comprise greater than about 95 wt-% 4-({[(2-methylpropanoyloxy)ethoxy]carbonylamino}methyl)cyclohexanecarboxylic acid are disclosed.

In a fourth aspect, methods of treating bleeding in a subject are disclosed comprising orally administering to a subject in need of such treatment at least one oral tablet dosage form provided by the present disclosure.

### BRIEF DESCRIPTION OF THE DRAWINGS

Those skilled in the art will understand that the drawings, described herein, are for illustration purposes only. The drawings are not intended to limit the scope of the present disclosure.

FIG. 1 shows dissolution profiles for tablets containing 4-({[(2-methylpropanoyloxy)ethoxy]carbonylamino}methyl)cyclohexanecarboxylic acid.

“Compound (1)” means the tranexamic acid prodrug (1), 4-({[(2-methylpropanoyloxy)ethoxy]carbonylamino}methyl)cyclohexanecarboxylic acid, pharmaceutically acceptable salts thereof, pharmaceutically acceptable solvates of any of the foregoing, and crystalline forms of any of the foregoing. Compound (1) refers to the racemate (±)-4-({[(2-methylpropanoyloxy)ethoxy]carbonylamino}methyl)cyclohexanecarboxylic acid, and/or either of the two enantiomers. Compound (1) is used interchangeably with tranexamic acid prodrug (1). In certain embodiments, tranexamic acid prodrug (1) is the free acid. In certain embodiments, tranexamic acid prodrug (1) is the sodium salt.

Unless specifically indicated, compound (1) encompasses all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan. For example, resolution of the enantiomers or diastereomers may be accomplished, for example, by conventional methods such as crystallization in the presence of a resolving agent, or chromatography, using, for example a chiral high-pressure liquid chromatography (HPLC) column.

Compound (1) may also exist in several tautomeric forms including the enol form, the keto form, and mixtures thereof. Accordingly, the chemical structures depicted herein encompass all possible tautomeric forms of the illustrated compounds. Compounds of the present disclosure also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compound (1) may exist in unsolvated forms as well as solvated forms, including hydrated forms and as N-oxides. In general, compound (1) may be the free acid, hydrated, solvated, N-oxides, or combinations of any of the foregoing. Compound (1) may exist in multiple crystalline, co-crystalline, or amorphous forms. Compound (1) includes pharmaceutically acceptable salts thereof, or pharmaceutically acceptable solvates of the free acid form of any of the foregoing, as well as crystalline forms of any of the foregoing.

Compound (1) also includes solvates. A solvate refers to a molecular complex of a compound with one or more solvent molecules in a stoichiometric or non-stoichiometric amount. Such solvent molecules are those commonly used in the pharmaceutical art, which are known to be innocuous to a subject, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

“Dosage form” refers to a form of a formulation that contains an amount of active agent or prodrug of an active agent, i.e., tranexamic acid prodrug (1), which can be administered to a subject to achieve a therapeutic effect. An oral dosage form is intended to be administered to a subject via the mouth and swallowed. A dose of a drug may include one or more dosage forms administered simultaneously or over a period of time.

“Subject” includes mammals, such as for example, humans.

“Pharmaceutically acceptable” refers to approved or approvable by a regulatory agency of a federal or a state government, listed in a U.S. Pharmacopeia, or listed in other generally recognized pharmacopeia for use in mammals, including humans.

“Pharmaceutically acceptable salt” refers to a salt of a compound such as compound (1) that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and (2) salts formed when an acidic proton present in the parent compound either is replaced by a metal ion, e.g., an alkali metal ion, an alkaline earth metal ion, or an aluminum ion; or coordinates with an organic base such as ethanolamine, diethanolamine, triethanolamine, N-methylglucamine, and the like. In certain embodiments, a salt of compound (1) is the hydrochloride salt, and in certain embodiments, the sodium salt.

“Pharmaceutically acceptable vehicle” or “pharmaceutically acceptable excipient” refers to a pharmaceutically acceptable diluent, a pharmaceutically acceptable adjuvant, a pharmaceutically acceptable excipient, a pharmaceutically acceptable carrier, or a combination of any of the foregoing with which a compound such as the tranexamic acid prodrug, 4-({[(2-methylpropanoyloxy)ethoxy]carbonylamino}methyl)cyclohexanecarboxylic acid (1), may be administered to a subject, which does not substantially compromise the pharmacological activity thereof, and which is nontoxic when administered in doses sufficient to provide a therapeutically effective amount of the tranexamic acid prodrug or tranexamic acid metabolite.

“Prodrug” refers to a derivative of an active compound (drug) that undergoes a transformation under the conditions of use, such as within the body, to release an active drug. Prodrugs are frequently, but not necessarily, pharmacologically inactive until converted into the active drug. Prodrugs may be obtained by bonding a promoiety (defined herein), typically via a functional group, to a drug. For example, tranexamic acid prodrug (1) is metabolized within a subject's body to form the parent drug tranexamic acid.

“Promoiety” refers to a group bonded to a drug, typically to a functional group of the drug, via a bond(s) that is cleavable under specified conditions of use. The bond(s) between the drug and promoiety may be cleaved by enzymatic or non-enzymatic means. Under the conditions of use, for example following administration to a subject, the bond(s) between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation of a subject to which the prodrug is administered or the acidic conditions of the stomach or the agent may be supplied exogenously. For example, for tranexamic acid prodrug (1), the drug is tranexamic acid and the promoiety has the structure:

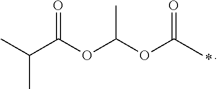

Consistent with “Dissolution Testing of Immediate Release Solid Oral Dosage Forms—Guidance for Industry”, FDA-CDER, August 1997, dissolution profiles may be considered similar based on a difference factor (f₁) and a similarity factor (f₂). For dissolution profiles to be considered similar, f₁ values should be close to 0, and f₂ values should be close to 100. Generally, f₁ values up to 15 (0-15) and f₂ values greater than 50 (50-100) ensure sameness or equivalence of two dissolution profiles. Procedures for calculating f₁ and f₂ are set forth in the foregoing reference.

“Sustained release” refers to release of a compound from a dosage form at a rate effective to achieve a therapeutic amount of tranexamic acid in the systemic blood circulation over a prolonged period of time relative to that achieved by oral administration of an immediate release formulation of tranexamic acid. In some embodiments, in vivo release of tranexamic acid occurs over a period of at least about 4 hours, in some embodiments, over a period of at least about 8 hours, in some embodiments over a period of at least about 12 hours, in some embodiments, over a period of at least about 16 hours, in some embodiments, over a period of at least about 20 hours, and in some embodiments, over a period of at least about 24 hours.

“Therapeutically effective amount” refers to the amount of 4-({[(2-methylpropanoyloxy)ethoxy]carbonylamino}methyl)cyclohexanecarboxylic acid that, when administered to a subject for treating bleeding, is sufficient to reduce, eliminate, or prevent bleeding in the subject. The therapeutically effective amount may vary depending, for example, on the compound, the cause of bleeding, the severity of the bleeding, the age, weight, and/or health of the subject to be treated, and the judgment of the prescribing physician. A therapeutically effective amount may be ascertained by those skilled in the art and/or capable of determination by routine experimentation.

“Treating” or “treatment” of bleeding refers to reducing, eliminating, or preventing bleeding in a subject experiencing bleeding or who anticipates a bleeding episode.

Reference is now made in detail to certain embodiments of dosage forms and methods. The disclosed embodiments are not intended to be limiting of the claims. To the contrary, the claims are intended to cover all alternatives, modifications, and equivalents.

Sustained release oral dosage forms provided by the present disclosure comprise 4-({[(2-methylpropanoyloxy)ethoxy]carbonylamino}methyl)cyclohexanecarboxylic acid and pharmaceutically acceptable excipients. 4-({[(2-Methylpropanoyloxy)ethoxy]carbonylamino}methyl)cyclohexanecarboxylic acid, as given by the following structure:

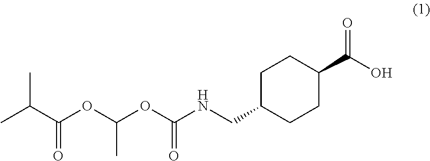

exhibits high oral bioavailability of tranexamic acid. Compound (1) includes 4-({[(2-methylpropanoyloxy)ethoxy]carbonylamino}methyl)cyclohexanecarboxylic acid and pharmaceutically acceptable salts thereof. In certain embodiments, compound (1) is the free acid form of 4-({[(2-methylpropanoyloxy)ethoxy]carbonylamino}methyl)cyclohexanecarboxylic acid. In certain embodiments, compound (1) is crystalline, and in certain embodiments, is the crystalline form of the free acid of 4-({[(2-methylpropanoyloxy)ethoxy]carbonylamino}methyl)cyclohexanecarboxylic acid.

Compound (1) may be prepared using the methods described by Gallop et al., U.S. Pat. No. 6,818,787, U.S. Pat. No. 7,186,855, U.S. Pat. Nos. 7,227,028, and 6,927,036; Estrada et al., U.S. Patent Application Publication No. 2005/0154057; Bhat et al., U.S. Patent Application Publication No. 2005/0070715; and/or Raillard et al., U.S. Pat. No. 7,332,924, and U.S. Patent Application Publication Nos. 2010/0087667 and 2010/0081830.

Sustained release oral dosage forms provided by the present disclosure comprise 4-({[(2-methylpropanoyloxy)ethoxy]carbonylamino}methyl)cyclohexanecarboxylic acid and one or more pharmaceutically acceptable excipients. Sustained release oral dosage forms may comprise greater than about 80 wt-% compound (1), greater than about 85 wt-% compound (1), greater than about 90 wt-% compound (1), or in certain embodiments, greater than about 95 wt-% compound (1), where wt-% is based on the total weight of a dosage form. In certain embodiments, an oral dosage from comprises from about 80 wt-% to about 95 wt-% compound (1), and in certain embodiments, about 80 wt-% to about 90 wt-% compound (1). In certain embodiments, a dosage form may contain from about 100 mg to about 2,000 mg compound (1), and in certain embodiments, about 500 mg compound (1) to about 1500 mg compound (1).

In certain embodiments, the one or more pharmaceutically acceptable excipients include hydroxypropylmethyl cellulose (HPMC) and a pharmaceutically acceptable lubricant. The amount of HPMC in a dosage form may be from about 5 wt-% to about 15 wt-%, from about 8 wt-% to about 12 wt-%, in certain embodiments, from about 9 wt-% to about 11 wt-%, and in certain embodiments is about 10 wt-% hydroxypropylmethyl cellulose. In certain embodiments, the hydroxypropylmethyl cellulose is chosen from a hypromellose 2208 polymer characterized by a methoxyl content from about 19% to about 24% and a hydroxypropyl content from about 7% to about 12% such as, for example, METHOCEL™ K3, METHOCEL™ K100, METHOCEL™ K4M, METHOCEL™ K15M, and METHOCEL™ K100M (Dow Chemical), or other chemically equivalent polymer. In certain embodiments, the hydroxypropylmethyl cellulose is a hypromellose 2208 polymer having a viscosity from about 3,000 cps to about 5,600 cps in a 2% aqueous solution such as METHOCEL™ K4M. In certain embodiments, the hydroxypropylmethyl cellulose is a hypromellose 2208 polymer having a methoxyl content from about 19% to about 24%, a hydroxypropyl content from about 7% to about 12%, and a viscosity from about 80,000 cps to about 120,000 cps in a 2% aqueous solution, such as METHOCEL™ K100M. In certain embodiments the hydroxypropylmethyl cellulose in a dosage form can be a combination of any of the above polymers.

The amount of lubricant in a dosage form may be from about 0.5 wt-% to about 4 wt-%, from about 1 wt-% to about 3 wt-%, and in certain embodiments is about 2 wt-%. In certain embodiments, the lubricant may be chosen from magnesium stearate, sodium stearyl fumarate, and stearic acid; and in certain embodiments, the lubricant is magnesium stearate.

Sustained release oral dosage forms provided by the present disclosure have a high loading of 4-({[(2-methylpropanoyloxy)ethoxy]carbonylamino}methyl)cyclohexanecarboxylic acid, e.g., greater than about 80 wt-% compound (1). In certain embodiments, the dosage form comprises granules, wherein the granules comprise the 4-({[(2-methylpropanoyloxy)ethoxy]carbonylamino}methyl)cyclohexanecarboxylic acid. In certain embodiments, compound (1) is provided in the form of granules having a high loading of compound (1). For example, the granules may comprise greater than about 95 wt-% compound (1), greater than about 96 wt-% compound (1), greater than about 97 wt-% compound (1), greater than about 98 wt-% compound (1), and in certain embodiments, greater than about 99 wt-% compound (1). In certain embodiments, the granules may further comprise a release rate-controlling polymer such as a hydroxypropylmethyl cellulose and a surfactant.

In certain embodiments, granules may comprise from about 0.5 wt-% to about 2.0 wt-%, in certain embodiments from about 0.5 wt-% to about 1.5 wt-%, and in certain embodiments about 1 wt-% of a polymer such as hydroxypropylmethyl cellulose, which may function as a release rate-controlling polymer and/or as a binder. The hydroxypropylmethyl cellulose may be chosen from a hypromellose 2910 characterized by a methoxyl content from about 28% to about 30% and a hydroxypropyl content from about 7% to about 12%, such as, for example, METHOCEL™ E3, METHOCEL™ E5, METHOCEL™ E6, METHOCEL™ E15, METHOCEL™ E50, METHOCEL™ E4M, METHOCEL™ E10M, METHOCEL™ A15 METHOCEL™ ES, METHOCEL™ E15, and METHOCEL™ K3 (Dow Chemical), or other chemically equivalent polymer. Other suitable hyddropypropyl methylcellulose polymers for use in matrix tablet formulations include METHOCEL™ E50LV, METHOCEL™ K100LV, METHOCEL™ K100LV CR, METHOCEL™ K4M, METHOCEL™ K15M, METHOCEL™ E4M, METHOCEL™ E10M, METHOCEL™K4MCR, METHOCEL™ K15MCR, METHOCEL™ K100MCR, METHOCEL™ E4MCR, and METHOCEL™ E10MCR (Dow Chemical). In certain embodiments, the METHOCEL™ will be for pharmaceutical use. e.g., Premium grade. In certain embodiments, the hydroxypropylmethyl cellulose is a hypromellose 2910 polymer having a viscosity from about 3,000 cps to about 5,600 cps in a 2% aqueous solution, such as METHOCEL™ E4M (HPMC K4M). In certain embodiments, the hydroxypropylmethyl cellulose is a hypromellose 2208 polymer having a methoxyl content from about 28% to about 30%, a hydroxypropyl content from about 7% to about 12%, and a viscosity from about 7,500 cps to about 14,000 cps in a 2% aqueous solution, such as METHOCEL™ E10M.

In certain embodiments, granules may comprise from about 0.5 wt-% to about 2.0 wt-% surfactant, from about 0.5 wt-% to about 1.5 wt-% surfactant or in certain embodiments, about 1 wt-% surfactant. A surfactant may be chosen from sodium lauryl sulfate, poloxamers (triblock copolymers of poly(propylene oxide) and poly(ethylene oxide)), and polysorbates (polyethylene derivatives of sorbitan monolaurate). In certain embodiments, the surfactant is sodium lauryl sulfate.

In certain embodiments, granules consist essentially of about 98 wt-% 4-({[(2-methylpropanoyloxy)ethoxy]carbonylamino}methyl)cyclohexanecarboxylic acid; about 1 wt-% of a surfactant; and about 1 wt-% a hypromellose 2910 polymer having a methoxyl content from about 28% to about 30%, a hydroxypropyl content from about 7% to about 12%, and a viscosity from about 3,000 cps to about 5,600 cps in a 2% aqueous solution. In certain embodiments, granules consist essentially of about 98 wt-% 4-({[(2-methylpropanoyloxy)ethoxy]carbonylamino}methyl)cyclohexanecarboxylic acid; about 1 wt-% of a surfactant; and about 1 wt-% METHOCEL™ E4M. In certain embodiments, granules consist essentially from about 97 wt-% to about 99 wt-% 4-({[(2-methylpropanoyloxy)ethoxy]carbonylamino}methyl)cyclohexanecarboxylic acid; from about 0.5 wt-% to about 1.5 wt % of a surfactant; and from about 0.5 wt-% to about 1.5 wt-% of a hypromellose 2208 polymer having a methoxyl content from about 28% to about 30%, a hydroxypropyl content from about 7% to about 12%, and a viscosity from about 7,500 cps to about 14,000 cps in a 2% aqueous solution. In certain embodiments, granules consist essentially of about 98 wt-% 4-({[(2-methylpropanoyloxy)ethoxy]carbonylamino}methyl)cyclohexanecarboxylic acid; about 1 wt-% of a surfactant; and about 1 wt-% METHOCEL™ E10M.

Granules having a high loading of compound (1) may be prepared using high shear wet granulation. At least in part, the amount of release rate-controlling polymer/binder and surfactant used to form the granules is chosen to provide a wide processing window for the amount of water used during granulation. For manufacturing, it is generally desirable to be able to vary process parameters without significantly negatively impacting the properties of the granules and to produce granules having optimal flow and mechanical properties to facilitate subsequent tableting processes. Thus, granules provided by the present disclosure may be prepared using a water equivalent from about 50 wt-% to about 75 wt-% of the dry formulation used to prepare the granules, from about 55 wt-% to about 70 wt-% of the dry formulation used to prepare the granules, and in certain embodiments, from about 60 wt-% to about 65 wt-% of the dry formulation used to prepare the granules.

Granules provided by the present disclosure, such as for example, granules comprising compound (1), METHOCEL™ E4M, and sodium lauryl sulfate, exhibit a Flodex Index less than about 10 mm, and in certain embodiments, less than about 5 mm.

When the constituents of the granules are incorporated into oral dosage forms, in certain embodiments, oral dosage forms provided by the present disclosure comprise from about 80 wt-% to about 90 wt-% compound (1), from about 0.8 wt-% to about 1.0 wt-% of a first hydroxypropylmethyl cellulose polymer, from about 0.8 wt-% to about 1.0 wt-% surfactant, from about 3 wt-% to about 15 wt-% of a second hydroxypropylmethyl cellulose polymer, and from about 0.5 wt-% to about 3.5 wt-% of a lubricant. In certain embodiments, oral dosage forms provided by the present disclosure comprise from about 85 wt-% to about 90 wt-% compound (1), from about 0.8 wt-% to about 1.0 wt-% of a hypromellose 2910 polymer having a methoxyl content from about 28% to about 30%, a hydroxypropyl content from about 7% to about 12%, and a viscosity from about 3,000 cps to about 5,600 cps in a 2% aqueous solution, from about 0.8 wt-% to about 1.0 wt-% sodium lauryl sulfate, from about 5 wt-% to about 15 wt-% of hypromellose 2208 polymer having a methoxyl content from about 19% to about 24%, a hydroxypropyl content from about 7% to about 12%, and a viscosity from about 80,000 cps to about 120,000 cps in a 2% aqueous solution, and from about 1.0 wt-% to about 3.0 wt-% of magnesium stearate. In certain embodiments, oral dosage forms provided by the present disclosure comprise from about 85 wt-% to about 90 wt-% compound (1), from about 0.8 wt-% to about 1.0 wt-% of a hypromellose 2910 polymer having a methoxyl content from about 28% to about 30%, a hydroxypropyl content from about 7% to about 12%, and a viscosity from about 3,000 cps to about 5,600 cps in a 2% aqueous solution, from about 0.8 wt-% to about 1.0 wt-% sodium lauryl sulfate, from about 5 wt-% to about 15 wt-% of hypromellose 2208 polymer having a methoxyl content from about 19% to about 24%, a hydroxypropyl content from about 7% to about 12%, and a viscosity from about 3,000 cps to about 5,600 cps in a 2% aqueous solution, and from about 1.0 wt-% to about 3.0 wt-% of magnesium stearate. In certain embodiments, oral dosage forms provided by the present disclosure comprise from about 85 wt-% to about 90 wt-% compound (1), from about 0.8 wt-% to about 1.0 wt-% of METHOCEL™-E4M, from about 0.8 wt-% to about 1.0 wt-% sodium lauryl sulfate, from about 5 wt-% to about 15 wt-% of METHOCEL™-K100M, and from about 2.0 wt-% to about 3.0 wt-% of magnesium stearate. In certain embodiments, oral dosage forms provided by the present disclosure comprise from about 85 wt-% to about 90 wt-% compound (1), from about 0.8 wt-% to about 1.0 wt-% of METHOCEL™-E4M, from about 0.8 wt-% to about 1.0 wt-% sodium lauryl sulfate, from about 5 wt-% to about 15 wt-% of METHOCELT™-K4M, and from about 2.0 wt-% to about 3.0 wt-% of magnesium stearate.

Sustained release oral dosage forms provided by the present disclosure may be provided as tablets. Formulations used to prepare the tablets comprise a blend of one or more pharmaceutically acceptable excipients and granules comprising a high loading of compound (1) and one or more pharmaceutically acceptable excipients. The granules are prepared by high shear wet granulation methods. Formulations provided by the present disclosure are generally useful in forming oral tablet dosage forms by direct compression.

In certain embodiments, dosage forms may be in the form of tablets comprising compound (1). Tablet dosage forms may be of any shape suitable for oral administration of a drug such as spheroidal, cube-shaped, oval, or ellipsoidal. In certain embodiments, tablet dosage forms, e.g., an oral dosage form in the form of a tablet, provided by the present disclosure are matrix systems in which the tranexamic acid prodrug (1) is dispersed in a matrix comprising at least one release-rate modifying compound. Matrix systems are well-known in the art as described, for example, in “Handbook of Pharmaceutical Controlled Release Technology,” ed. Wise, Marcel Dekker, Inc. (2000) and “Treatise on Controlled Drug Delivery, Fundamentals, Optimization, and Applications,” ed. Kydonieus, Marcel Dekker, Inc. (1992).

In certain embodiments, the amount of compound (1) in a dosage form provided by the present disclosure is from about 100 mg to about 2,000 mg, in certain embodiments, from about 500 mg to about 1,500 mg, and in certain embodiments is about 300 mg, about 600 mg, about 900 mg, or about 1,200 mg. For dosage forms comprising a pharmaceutically acceptable salt and/or solvate of compound (1), the amount of compound (1) in a dosage form refers to the mass equivalent weight of compound (1) comprising the salt and/or hydrate. In certain embodiments, tablet dosage forms may comprise a therapeutically effective amount of compound (1). A therapeutically effective amount of compound (1) may comprise from about 50 mg-equivalents to about 1,000 mg-equivalents tranexamic acid, and in certain embodiments from about 250 mg-equivalents to about 750 mg-equivalents tranexamic acid. For reference, one (1) mg compound (1) corresponds to 0.479 mg-equivalents of tranexamic acid.

In certain embodiments in which tablet dosage forms comprise less than a therapeutically effective amount of compound (1), multiple tablet dosage forms may be administered to a subject simultaneously or over a period of time to provide a therapeutically effective dose of compound (1).

In addition to compound (1) and a release rate modifying compounds disclosed herein, tablet dosage forms may also comprise one or more pharmaceutically acceptable vehicles such as surfactants, lubricants, plasticizers, binding agents, diluents, anti-adherents, glidants, buffers, dyes, wetting agents, emulsifying agents, pH buffering agents, stabilizing agents, thickening agents, disintegrants, and coloring agents.

Diluents, or fillers, may be added to increase the bulk to make dosage forms a practical size for compression. Examples of diluents useful in tablet dosage forms provided by the present disclosure include dibasic calcium phosphate-dibasic calcium phosphate dihydrate, calcium sulfate, dicalcium phosphate, tricalcium phosphate, lactose, cellulose including microcrystalline cellulose, kaolin, mannitol, sodium chloride, dry starch, pregelatinized starch, compressible sugar, and combinations of any of the foregoing. In certain embodiments, a diluent is selected from dibasic calcium phosphate and microcrystalline cellulose. Fillers may be water insoluble, water soluble, or combinations thereof. Examples of useful water insoluble fillers include silicon dioxide, titanium dioxide, talc, alumina, starch, kaolin, polacrilin potassium, powdered cellulose, microcrystalline cellulose, fumed silica, glyceryl monostearate, magnesium stearate, calcium stearate, colloidal silica, micronized silica, magnesium trisilicate, gypsum, and combinations of any of the foregoing. Examples of water-soluble fillers include water soluble sugars and sugar alcohols, such as lactose, glucose, fructose, sucrose, mannose, dextrose, galactose, the corresponding sugar alcohols and other sugar alcohols, such as mannitol, sorbitol, xylitol, and combinations of any of the foregoing.

Glidants may be included in dosage forms provided by the present disclosure to reduce sticking effects during processing, film formation, and/or drying. Examples of useful glidants include talc, magnesium stearate, glycerol monostearate, colloidal silicon dioxide, precipitated silicon dioxide, fumed silicon dioxide, and combinations of any of the foregoing. In certain embodiments, a glidant is colloidal silicon dioxide.

Binding agents may be included in dosage forms to facilitate adhesion of the constituents. Examples of binding agents useful in tablet dosage forms provided by the present disclosure include polyvinyl acetate phthalate, molasses, methylcellulose, hydroxypropyl cellulose (HPC), hydroxypropyl methyl cellulose (HPMC), sodium carboxymethyl cellulose, microcrystalline cellulose, and polyvinyl pyrrolidone. In certain embodiments provided by the present disclosure, a binder is microcrystalline cellulose such as MCC PH200 (Avicel®, FMC Corporation).

Plasticizers may be included in tablet dosage forms provided by the present disclosure. Examples of plasticizers useful in tablet dosage forms provided by the present disclosure include alkyl citrates such as triethyl citrate, acetyl triethyl citrate, tributyl citrate, acetyl triethyl citrate, and acetyl tributyl citrate; alkyl acetates such as triethyl acetate, acetyl triethyl acetate, tributyl acetate, acetyl triethyl acetate, and acetyl tributyl acetate; sucrose fatty acid esters; glycerin mono-, di- and tri-fatty acid esters such as triacetin, glycerin mono-fatty acid esters, glycerin monostearate and acetylated monoglyceride; polyglycerin fatty acid esters; polyethylene glycols such as macrogol 400, macrogol 600, macrogol 1500, macrogol 4000, macrogol 6000, macrogol 20,000, and macrogol 35,000; dibutyl sebacate; tributyl sebacate; vinyl pyrrolidone; propylene glycol; sesame oil; castor oil; glycerin; silicone resins; D-sorbitol; phytosterol; alkyl phthalates such as diethyl phthalate, dibutyl phthalate and dioctyl phthalate; adipate polyesters; isopropyl myristate; medium chain triglyceride; butyl phthalyl butyl glycolate; polyoxyethylene polyoxypropylene glycol; and combinations of any of the foregoing.

Lubricants and anti-adherents may be included in tablet dosage forms provided by the present disclosure to aid in processing. Examples of lubricants and/or anti-adherents useful in tablet dosage forms provided by the present disclosure include calcium stearate, glyceryl behenate, glyceryl monostearate, magnesium stearate, mineral oil, polyethylene glycol, sodium stearyl fumarate, sodium lauryl sulfate, sodium dodecyl sulfate, stearic acid, talc, hydrogenated vegetable oil, zinc stearate, and combinations of any of the foregoing. In certain embodiments, a lubricant is glyceryl monostearate. In certain embodiments, a lubricant is magnesium stearate.

Examples of surfactants useful in tablet dosage forms provided by the present disclosure include pharmaceutically acceptable anionic surfactants, cationic surfactants, zwitterionic, amphoteric (amphiphatic/amphiphilic) surfactants, non-ionic surfactants, polyethyleneglycol esters or ethers, and combinations of any of the foregoing. Examples of useful pharmaceutically acceptable anionic surfactants include monovalent alkyl carboxylates, acyl lactylates, alkyl ether carboxylates, N-acyl sarcosinates, polyvalent alkyl carbonates, N-acyl glutamates, fatty acid-polypeptide condensates, sulfuric acid esters, alkyl sulfates such as sodium lauryl sulfate and sodium dodecyl sulfate, ethoxylated alkyl sulfates, ester linked sulfonates such as docusate sodium and dioctyl sodium succinate, alpha olefin sulfonates, or phosphated ethoxylated alcohols. Examples of useful pharmaceutically acceptable cationic surfactants include monoalkyl quaternary ammonium salts, dialkyl quaternary ammonium compounds, amidoamines, and aminimides. Examples of useful pharmaceutically acceptable amphoteric surfactants include N-substituted alkyl amides, N-alkyl betaines, sulfobetaines, and N-alkyl-6-aminopropionates. Examples of useful pharmaceutically acceptable nonioinic surfactants include diblock and triblock copolymers of polyethylene oxide, polypropylene oxide, polyoxyethylene (20) sorbitan monooleate, and polyethyleneglycol esters or ethers such as polyethoxylated castor oil, polyethoxylated hydrogenated castor oil, and hydrogenated castor oil. In certain embodiments, a surfactant is chosen from sodium lauryl sulfate and sodium dodecyl sulfate.

Disintegrants may be included in a tablet formulation to cause a tablet to break apart, for example, by expansion of a disintegrant when exposed to water. Examples of useful disintegrants include water swellable substances such as low-substituted hydroxypropyl cellulose, cross-linked sodium carboxymethylcellulose (sodium croscarmellose), sodium starch glycolate, sodium carboxymethylcellulose, sodium carboxymethyl starch, ion-exchange resins, microcrystalline cellulose, cross-linked polyvinyl pyrrolidone, starches and pregelatinized starch, formalin-casein, alginic acid, certain complex silicates, and combinations of any of the foregoing.

Tablet dosage forms provided by the present disclosure may further comprise one or more coatings, which may partially or fully cover the tablets. While certain coatings may be applied to modify or affect the release of compound (1) from a tablet dosage form in the gastrointestinal tract, others may have no such effect. For example, one or more additional coatings may be for physical protection, aesthetics, ease in swallowing, identification, and/or to facilitate further processing of the tablets. Coatings may be impermeable to moisture or moisture permeable. Moisture impermeable exterior tablet coatings may be useful for maintaining low moisture content in a dosage form that is packaged in the presence of a desiccant and may thereby enhance, for example, the storage stability of a tablet dosage form. Examples of materials useful in coatings for physical protection include permeable or soluble materials such as hydroxypropyl methylcellulose, hydroxypropyl cellulose, lactose, hydroxypropyl ethylcellulose, hydroxyethyl cellulose, and xanthan gum. Examples of materials useful in external tablet coatings to facilitate further processing include talc, colloidal silica, polyvinyl alcohol, titanium dioxide, micronized silica, fumed silica, glycerol monostearate, magnesium trisilicate, and magnesium stearate. An external tablet coating may further include one or more vehicles such as plasticizers, binders, fillers, lubricants, compression aides, and combinations of any of the foregoing. The one or more additional coatings may comprise a single material or a combination of more than one material including any of those disclosed herein. These additional coatings may be applied to tablet dosage forms by methods known to those skilled in the art.

In certain embodiments, an oral dosage form comprises a granulation comprising greater than about 95 wt-% 4-({[(2-methylpropanoyloxy)ethoxy]carbonylamino}methyl)cyclohexanecarboxylic acid, and in certain embodiments greater than about 97 wt-% 4-({[(2-methylpropanoyloxy)ethoxy]carbonylamino}methyl)cyclohexanecarboxylic acid. In certain embodiments, an oral dosage form comprising a granulation may be compressed into a tablet dosage form. In certain embodiments, an oral dosage form comprising a granulation may be inserted into and contained in a capsule dosage form. In certain embodiments, an oral dosage form comprising a granulation may be a liquid oral dosage from such as an emulsion or suspension.

The release characteristics of dosage forms provided by the present disclosure comprising compound (1) may be characterized, in part, by the in vitro dissolution profile. Methods for determining dissolution profiles of dosage forms are well known to those skilled in the pharmaceutical arts. Standard methodologies set forth in the U.S. Pharmacopeia may be used. For example, a dissolution profile may be determined using either a U.S. Pharmacopeia Type I Apparatus (baskets) or a U.S. Pharmacopeia Type II Apparatus (paddles).

Using the latter method, dissolution, or release, profiles of dosage forms provided by the present disclosure may be determined by immersing the dosage forms in a 10 mM potassium phosphate monobasic buffer (KH₂PO₄) at pH 7.4, with 1%-vol SLS and a temperature of 37° C. The dissolution medium is stirred at 50 rpm (USP, Type II). Samples are withdrawn from the dissolution medium at intervals and the content of compound (1) in the dissolution medium determined using reverse phase high pressure liquid chromatography (HPLC).

In certain embodiments, release of compound (1) from tablet dosage forms provided by the present disclosure exhibits an in vitro dissolution profile in 10 mM, pH 7.4, potassium phosphate monobasic buffer with 1% sodium lauryl sulfate at 37° C. stirred at 50 rpm (USP, Type II) wherein about 20% to about 45% of the 4-({[(2-methylpropanoyloxy)ethoxy]carbonylamino}methyl)cyclohexanecarboxylic acid in the dosage form is released within about 4 hours; about 40% to about 70% of the 4-({[(2-methylpropanoyloxy)ethoxy]carbonylamino}methyl)cyclohexanecarboxylic acid is released within about 8 hours; about 60% to about 85% of the 4-({[(2-methylpropanoyloxy)ethoxy]carbonylamino}methyl)cyclohexanecarboxylic acid is released within about 12 hours; and about 80% to about 100% of the 4-({[(2-methylpropanoyloxy)ethoxy]carbonylamino}methyl)cyclohexanecarboxylic acid is released within about 20 hours.

In certain embodiments, a tablet exhibits a dissolution profile that is similar to the foregoing profile as determined using the f1 difference factor and the f2 similarity factor according to FDA guidelines.

In certain of such embodiments, a tablet dosage form exhibiting the foregoing release profiles comprises about 900 mg or about 1,000 mg compound (1).

It is generally recognized that commercially acceptable tablets have a friability of less than about 1 wt-% determined according to USP Test No. 1216. In certain embodiments, tablets provided by the present disclosure have a friability of less than about 1 wt-%, in certain embodiments, less than about 0.5 wt-%, in certain embodiments, less than about 0.3 wt-%, and in certain embodiments, less than about 0.1 wt-%.

Sustained release oral dosage forms provided by the present disclosure may be administered to a subject having a disease, disorder, or condition for which tranexamic acid is known, believed to be, or hereafter determined to be therapeutically effective. For example, tranexamic acid is known to be or is expected to be useful in treating bleeding. Therefore, dosage forms provided by the present disclosure are expected to be useful in treating bleeding such as perioperative bleeding, gastrointestinal bleeding, drug-induced bleeding, bleeding caused by a wound, bleeding caused by a combat injury, bleeding associated with cancer, bleeding during dental procedures, menorrhagia, bleeding associated with biopsy, bleeding associated with dialysis, and bleeding caused by a bleeding disorder. In certain embodiments, oral tablet dosage forms provided by the present disclosure may be used to treat menorrhagia, in certain embodiments, gastrointestinal bleeding, in certain embodiments perioperative bleeding, and in certain embodiments bleeding caused by a wound. In certain embodiments, compound (1) may be used to treat bleeding in a subject with a bleeding disorder. In certain embodiments compound (1) may be administered to the subject at least one hour prior to an anticipated bleeding episode.

The suitability of dosage forms provided by the present disclosure to treat bleeding may be determined by methods described in the art.

A suitable dose of compound (1) to be administered to a subject in need of tranexamic acid therapy may be estimated based on the mass equivalent of tranexamic acid and the oral bioavailability of tranexamic acid provided by compound (1).

Dosage forms provided by the present disclosure may be administered to reduce or minimize bleeding in a subject who either anticipates bleeding such as during surgery or traumatic injury or who is bleeding. A dosage form provided by the present disclosure may be effectively used prophylactically to reduce or minimize bleeding such as perioperative bleeding and in bleeding due to traumatic injury.

Bleeding refers to extravasation of blood from any component of the circulatory system and includes unwanted and uncontrolled bleeding in connection with surgery, trauma, or other forms of tissue damage, as well as unwanted bleedings in subjects having bleeding disorders. Bleeding may occur in subjects having a basically normal coagulation system but who are experiencing a (temporary) coagulopathy, as well as in subjects having congenital or acquired coagulation bleeding disorders. Dosage forms provided by the present disclosure may be used to control bleeding in subjects having a bleeding disorder or may be used to control bleeding occurring in subjects with a normally functioning blood clotting cascade (no clotting factor deficiencies or inhibitors against any of the coagulation factors).

Subjects administered dosage forms provided by the present disclosure perioperatively may or may not have an underlying bleeding disorder.

Menorrhagia is defined as blood loss >80 mL per menstrual cycle and affects many women and represents a significant health problem. Prevalence rates are believed to be similar across the Western world, and in the U.K. at least one in 20 women aged between 34 and 49 years will consult their general practitioners because of menstrual disorders. Menorrhagia accounts for 60% of primary-care consultations for menstrual problems and 12% of all gynecology referrals (Peto et al., Fam. Pract., 1993, 10, 207-211; McPherson and Andersson, eds., Women's problems in general practice, Oxford: Oxford University Press, 1983, pp 21-41; Bradlow et al., Patterns of referral, Oxford: Oxford Health Services Research Unit, 1992). While various pathological mechanisms may contribute to the cause of menorrhagia, approximately 50% of women with heavy menstrual blood loss have no underlying anatomical or endocrinological abnormality. In such women fibrinolytic activity in utero is higher than in women with normal menstrual blood loss, with this increased fibrinolysis resulting from elevated levels of endometrium-derived plasmin and plasminogen activators (Gleeson, Am. J. Obstet. Gynecol., 1994, 171, 178-183; Dockeray et al., Eur. J. Obstet. Gynecol. Reprod. Biol., 1987, 24, 309-318). Tranexamic acid is known to be useful in treating menorrhagia (Wellington and Wagstaff, Drugs 2003, 63(13), 1417-33).

In the surgical setting, skillful surgery combined with blood saving methods and careful management of blood coagulation can reduce unnecessary blood loss and transfusion requirements. Some surgical procedures may be associated with blood loss and/or compromised hemostasis in a subject without pre-existing hemostatic abnormalities. Typical surgical procedures that may be associated with hyperfibrinolysis include operations requiring cardiopulmonary bypass, orthotopic liver transplantation, and some urological and orthopedic operations. Moreover, there are subgroups of subjects who refuse blood transfusion in subjects with borderline or mild hemostatic defects such as subjects on antiplatelet agents or anticoagulants, subject with hepatic cirrhosis, and those with chronic renal failures. Excessive surgical bleeding causes hypovolanemia, hemodynamic instability, anemia and reduced oxygen delivery to tissues with a subsequent increase in postoperative morbidity and mortality. Adverse effects of allogeneic blood transfusion include transmission of infections, diseases, immunosuppression, transfusion-related acute lung injury, transfusion reactions, and graft-vs-host reactions. The cost implication for blood transfusion is also significant and includes the direct blood transfusion costs as well as indirect costs originating from additional treatments and prolonged hospitalization.

Systemic antifibrinolytic agents are widely used in major surgery to prevent fibrinolysis and thus reduce surgical blood loss (Mandy and Webster, Br. J. Anaesthesia 2004, 93(6), 842-58). A recent systematic review of randomized, controlled trials of antifibrinolytic agents in elective surgical subjects showed that perioperative administration of antifibrinolytic agents reduced the numbers needing transfusion by one third, reduced the volume needed per transfusion by one unit, and halved the need for further surgery to control bleeding (Henry et al., Cochrane Review, 2004). Tranexamic acid has been shown to be effective in reducing operative bleeding and/or post-operative bleeding in nasal surgery (Yaniv et al., Am J Rhinoplasty 2006, 20(2), 227-229); knee replacement surgery (Zohar et al., Anesth Analg 2004, 99, 1679-83); total knee arthroplasty (Lozano et al., Vox Sanguinis 2008, 95, 39-44; Cid and Lozano, Transfusion 2005, 45, 1302-1307); hip arthroplasty (Rosencher et al., Transfusion 2003, 43, 459-469); spinal fusion surgery (Wong et al., Anesth Analg 2008, 107, 1479-86); scoliosis surgery (Neilipovitz et al., Anesth Analg 2001, 93, 82-7); complex spine surgery (Colomina et al., Orthopedics 2009, 32(2), 91); orthopedic surgery (Zufferey et al., Anesthesiology 2006, 105, 1034-1046); cardiac surgery (Laupacis et al., Anesth Analg 1997, 85, 1258); orthognathic surgery (Choi et al., J Oral Maxillofac Surg 2009, 67, 125-133); coronary artery bypass surgery (Taghaddomi et al., J Cardiothroacic Vascular Anesthesia 2008); and prostatic surgery (Dunn and Goa, Drugs 1999, 57, 1005-32). Prophylactic administration of lysine analogs such as tranexamic acid has been shown to reduce post-operative bleeding in cardiopulmonary bypass surgery by 30-40% (Fremes et al., Ann Thorac Surg 1994, 58, 1580-8; and Levi et al., Lancet 1999, 354, 1940-7); reduce total blood loss in subjects undergoing total knee arthroplasty by up to 50% and decreased transfusion requirements without increasing the risk of thromboembolic manifestations (Hiippla et al., Anesth Analg 1997, 84, 839-44; Jansen et al., Br. J. Anaesth 1999, 83, 596-601; and Veien et al., Acta Anaesthesiol Scand 2002, 46, 1206-11); reduce intraoperative blood loss in subjects undergoing total hip replacement (Ekback et al., Anesth Analg 2000, 91, 1124-30; and Benoni et al., Acta Orthop Scand 2001, 72, 442-8); and reduce blood loss and transfusion requirements in orthotopic liver transplantation (Boylan et al., Anesthesiolgy 1996, 85, 1043-8; and Dalmau et al., Anesth Analg 2000, 91, 29-34 20).

In general, systemic antifibrinolytics such as tranexamic acid appear to be more effective in reducing bleeding when used prophylactically.

In certain embodiments, dosage forms provided by the present disclosure may be used to treat bleeding resulting from surgery. Examples of surgical procedures in which methods provided by the present disclosure can be useful include nasal surgery such as rhinoplasty, septoplasty, turbinectomy and functional endoscopy sinus surgery; orthognathic surgery; prostatectomy; splenectomy; gall bladder surgery; gynecological surgery such as oophorectomy, Cesarean section, and hysterectomy; liver transplant; eye surgery; dental surgery; laparoscopic surgery; cancer surgery including bladder cancer, lung cancer, and esophageal cancer; orthopedic surgery such as hip replacement, spinal fusion surgery, spinal surgery, scoliosis surgery, hip arthroplasty, and knee arthroplasty; and cardiac surgery such as coronary artery bypass surgery, and valve replacement surgery.

In certain embodiments, dosage forms provided by the present disclosure may be administered perioperatively to a subject including before surgery, during surgery, and/or after surgery.

In certain embodiments, dosage forms provided by the present disclosure may be administered prophylactically, before surgery, to treat bleeding during and/or following surgery (i.e., perioperative bleeding). In certain embodiments, prophylactic amounts of a compound of compound (1) may be administered from about 1 to about 24 hours before surgery; from about 1 to about 12 hours before surgery; from about 1 to about 6 hours before surgery; and in certain embodiments, from about 1 to about 3 hours before surgery. In certain embodiments, prophylactic amounts of compound (1) may be administered from about 1 day to about 3 days before surgery; from about 1 day to about 2 days before surgery; and in certain embodiments, about 1 day before surgery. In certain embodiments, a dosage form provided by the present disclosure may be administered at least about 2 hours before surgery, at least about 6 hours before surgery, at least about 12 hours before surgery, and in certain embodiments, at least about 24 hours before surgery.

Traumatic hemorrhage is the leading cause of death from wounds in the battlefield and the second leading cause of death in civilian trauma (Kauvar and Wade, Critical Care 2005, 9 (Suppl 5), S1-S9). Responses to trauma and subsequent resuscitation may include hypothermia, hemodilution and acidosis, conditions which can induce coagulopathies in which normal coagulation function is altered and disrupted. Approximately 20% of hemorrhagic deaths are due to compressible wounds (i.e., those that are accessible to direct pressure), treatable with pressure dressings, tourniquets, and mechanical surgical methods. However, the majority (approximately 80%) of hemorrhagic deaths on the battlefield are due to intracavitary hemorrhage, which is not accessible for direct compression such as within the pelvic, abdominal or thoracic cavities (Ryan et al., RTO-MP-HFM-109 2004). Currently, no method other than surgical intervention can treat intracavitary hemorrhage. In a controlled trial in which antifibrinolytic agents were administered following traumatic injury the results were inconclusive (Coats et al., Cochrane Database Syst Rev 2004, CD004896).

In certain embodiments, dosage forms provided by the present disclosure may be administered to a subject to treat bleeding caused by a wound prior to incurring the wound. In certain embodiments, a therapeutically effective amount of compound (1) may be administered to a subject to treat bleeding caused by a wound at least about 1 hour, at least about 2 hours, at least about 4 hours, at least about 8 hours, at least about 12, hours, or at least about 24 hours prior to incurring the wound. A compound may be administered prior to incurring an intentional wound such as, for example, prior to elective surgery, or prior to incurring an unintentional wound such, for example, prior to traumatic injury.

In certain embodiments, dosage forms provided by the present disclosure may be used to treat bleeding resulting from trauma. Traumatic injury includes, for example, abrasions, contusions, lacerations, incisions, gunshot wounds, blunt impact, and injury resulting from combat, including military combat and combat associated with law enforcement such as by police.

In certain embodiments, dosage forms provided by the present disclosure may be administered prophylactically to treat bleeding in anticipation of possible trauma, such as prior to combat. For example, in military situations in which combat is anticipated and the potential for traumatic injury is significant, a dosage form may be administered to the combatant at least about 2 hours, at least about 6 hours, at least about 12 hours, or at least about 24 hours prior to entering a combat situation. A compound of Formula (I) may continue to be administered as long as a significant potential for traumatic injury exists and as appropriate to provide a prophylactically effective plasma or blood concentration of tranexamic acid.

In certain embodiments, dosage forms provided by the present disclosure may be used to treat bleeding associated with bleeding disorders (Greaves and Watson, J Thrombosis Hemostasis 2007, 5(Suppl. 1), 167-174). A bleeding disorder can be any physiological defect of cellular or molecular origin that results in abnormal or pathological bleeding. A bleeding disorder may be congenital, acquired, or induced. Acquired bleeding disorders of primary hemostasis include bleeding due to pharmacological platelet inhibitors; clotting factor deficiencies such as hemophilia A, hemophilia B, hemophilia C, or deficiency of coagulation factors VII, IX, or XI; defective platelet function such as Glanzmann thombasthenia and Bernard-Doulier syndrome; thrombocytopenias; primary bone marrow diseases such as myeloproliferative, myelodysplastic, leukemic, and plasma cell dyscrsias; and severe renal failure. Acquired bleeding disorders of coagulation/fibrinolysis include hepatocellular failure, vitamin K deficiency, bleeding due to pharmacological anticoagulants, and coagulation factor inhibitors. Scurvy is another example of a mild bleeding disorder. Bleeding disorders include coagulopathy such as caused by a dilution of coagulation proteins, increased fibrinolysis and lowered number of platelets due to bleedings and/or tranfusions, such as in subjects having multiple transfusions. Bleeding disorders further include inherited macrothrombocytopenias (platelet disorders) such as Bernard-Soulier syndrome, MYH9 gene-related disorders, macrothrobocytopenia and 22q11.2 deletion syndrome, gray platelet syndrome, Montreal platelet syndrome, benign Mediterranean macrothrobocytopenia, macrothrobocytopenia associated with mitral valve insufficiency, macrothrombocytopenia with platelet expression of glycophorin A, and macrothrombocytopenia with neutropenia.

In certain embodiments, dosage forms provided by the present disclosure may be administered to treat hereditary thrombocytopenia syndromes including congenital amegakaryocytic thromobcytopeina (CAMT), thrombocytopenia absent radius syndrome, Fanconi anemia, Bernard-Soulier syndrome, May Hegglin anomaly, Grey platelet syndrome, or Alport syndrome.

In certain embodiments, dosage forms provided by the present disclosure may be administered to treat thrombocytopenia induced by valproic acid, methotrexate, carboplatin, interferon, isotetinoin, H2 blockers, chemotherapeutic agents, or proton pump inhibitors.

In certain embodiments, dosage forms provided by the present disclosure may be administered to treat thrombocytopenia characterized by increased platelet destruction such as idiopathic thrombocytopenic purpura, throbotic thrombocytopenic purpura, hemolytic-uremic syndrome, disseminated intravascular coagulation, paroxysmal nocturnal hemoglobinuria, antiphospholipid syndrome, systemic lupus erythematosus, post transfusion purpura, neonatal alloimmune thrombocytopenia, hypersplenism, Dengue fever, or HIV-associated thrombocytopenia.

In certain embodiments, dosage forms provided by the present disclosure may be administered to treat thrombocytopenia characterized by decreased platelet production including vitamin B12 or folic acid deficiency, leukemia or myelodysplastic syndrome, liver failure, sepsis and systemic viral or bacterial infection, and Dengue fever.

In certain embodiments, dosage forms provided by the present disclosure may be administered to treat gastrointestinal bleeding such as upper gastrointestinal bleeding, ulcerative colitis, or hemorrhagic gastritis.

In certain embodiments, dosage forms provided by the present disclosure may be administered to treat diffuse bleeding such uterine bleeding.

In certain embodiments, dosage forms provided by the present disclosure may be administered to treat bleeding associated with child birth including post partum hemorrhage.

In certain embodiments, dosage forms provided by the present disclosure may be administered to treat intracavitary bleeding (bleeding that occurs in organs) such as bleeding in the brain, inner ear, or eyes.

In certain embodiments, dosage forms provided by the present disclosure may be administered to treat bleeding in organs and tissue where there is limited ability to apply mechanical or surgical hemostasis.

In certain embodiments, dosage forms provided by the present disclosure may be administered to treat bleeding associated with surgery or trauma in subjects having acute hemarthroses (bleedings in joints), chronic hemophilic arthropathy, hematomas, (e.g., muscular, retroperitoneal, sublingual and retropharyngeal), bleedings in other tissue, hematuria (bleeding from the renal tract), cerebral hemorrhage, surgery (e.g., hepatectomy), dental extraction, and gastrointestinal bleedings.

Dosage forms provided by the present disclosure may be used to treat drug-induced bleeding. For example, bleeding may occur in subjects on anticoagulant therapy in whom a defective hemostasis has been induced by the therapy given. Anticoagulant therapy can be given to prevent thromboembolic disease and can include administration of heparin, other forms of proteoglycans, warfarin or other forms of vitamin K-antagonists as well as aspirin and other platelet aggregation inhibitors, such as, for example, antibodies or other inhibitors of GP IIb/IIIa activity. Bleeding may also be due to thrombolytic therapy which involves combined treatment with an antiplatelet agent (e.g., acetylsalicylic acid), an anticoagulant (e.g., heparin), and a fibrinolytic agent (e.g., tissue plasminogen activator, tPA).

In certain embodiments, dosage forms provided by the present disclosure may be administered to increase ultrafiltration capacity in dialysis (Kuriyama et al., Peritoneal Dialysis International 1999, 19(1), 38-44).

In certain embodiments, a dosage form provided by the present disclosure may be administered to treat bleeding during and after biopsy including, for example, liver biopsy, kidney biopsy, lung biopsy, tumor biopsy, gastrointestinal biopsy, and cervical conization.

In certain embodiments, dosage forms provided by the present disclosure may be administered to restore and/or promote hemostasis in a subject. Hemostasis refers to the physiologic process whereby bleeding is halted. Hemostatic agents are those that prevent, treat or ameliorate abnormal bleeding, such as abnormal bleeding caused by a bleeding disorder or bleeding episode. Disorders of hemostasis include, for example, platelet disorders, such as idiopathic thrombocytopenic purpura, and disorders of coagulation such as hemophilia. Hemostasis can also refer to the complex interaction between vessels, platelets, coagulation factors, coagulation inhibitors and fibrinolytic proteins to maintain the blood within the vascular compartment in a fluid state. The objective of the hemostatic system is to preserve intravascular integrity by achieving a balance between hemorrhage and thrombosis. Promoting hemostasis refers to the process of contributing to or improving hemostasis in a subject. For example, an agent that promotes hemostasis can be an agent that reduces abnormal bleeding, such as by halting bleeding more rapidly, or by reducing the amount of blood loss.

In certain embodiments, dosage forms provided by the present disclosure may be administered to a subject having a skin disease or disorder such as wound healing, epidermal hyperplasia, skin roughening, or unwanted skin pigmentation.

In certain embodiments, a dosage form provided by the present disclosure may be administered to a subject to treat bleeding associated with cancer or tumor metastasis (Bennett et al., Br J Haematol 1997, 99(3), 570-4).

When used to treat bleeding in a subject a therapeutically effective amount of compound (1) may be administered or applied singly, or in combination with other agents including other antifibrinolytic agents. Dosage forms provided by the present disclosure may also deliver compound (1) in combination with another pharmaceutically active agent.

Dosage forms, upon releasing tranexamic acid prodrug (1), provide tranexamic acid to a subject. The promoiety of compound (1) may be cleaved either chemically and/or enzymatically. One or more enzymes present in the stomach, intestinal lumen, intestinal tissue, blood, liver, brain or any other suitable tissue of a mammal can enzymatically cleave the promoiety or promoieties of the prodrug. If the promoiety is cleaved after absorption by the gastrointestinal tract, compound (1) can be absorbed into the systemic circulation from the large intestine. In certain embodiments, the promoiety is cleaved after absorption by the gastrointestinal tract. In certain embodiments, the promoiety or promoieties are cleaved in the gastrointestinal tract and tranexamic acid is absorbed into the systemic circulation form the large intestine. In certain embodiments, the tranexamic acid prodrug is absorbed into the systemic circulation from the gastrointestinal tract, and the promoiety is cleaved in the systemic circulation, after absorption of the tranexamic acid prodrug from the gastrointestinal tract.

It is believed that tablet dosage forms providing sustained systemic concentrations of tranexamic acid will enhance subject compliance as compared to the non-prodrug form which is currently administered up to six times per day, a regimen that is inconvenient for subjects and difficult for subjects to remember. Additionally, it is believed that the use of tablet oral dosage forms provided by the present disclosure will provide enhanced efficacy with reduced side effects which side effects may include dizziness, somnolence, fatigue, and/or ataxia.

The amount of 4-({[(2-methylpropanoyloxy)ethoxy]carbonylamino}methyl)cyclohexanecarboxylic acid that will be effective in the treatment of bleeding will depend, at least in part, on the nature of the disease, and may be determined by standard clinical techniques known in the art. In addition, in vitro or in vivo assays may be employed to help identify optimal dosing ranges. Dosing regimens and dosing intervals may also be determined by methods known to those skilled in the art. The amount of 4-({[(2-methylpropanoyloxy)ethoxy]carbonylamino}methyl)cyclohexanecarboxylic acid administered may depend on, among other factors, the subject being treated, the weight of the subject, the severity of the disease, the route of administration, and the judgment of the prescribing physician.

For systemic administration, a therapeutically effective dose may be estimated initially from in vitro assays. Initial doses may also be estimated from in vivo data, e.g., animal models, using techniques that are known in the art. Such information may be used to more accurately determine useful doses in humans. One having ordinary skill in the art may optimize administration to humans based on animal data.

A dose of 4-({[(2-methylpropanoyloxy)ethoxy]carbonylamino}methyl)cyclohexanecarboxylic acid can be adjusted to provide an equivalent molar quantity or mass equivalent dose of tranexamic acid. A dose can comprise multiple dosage forms provided by the present disclosure. Therapeutically effective doses of tranexamic acid in pediatric subjects are from about 25 mg to about 50 mg per kilogram body weight per day. In certain embodiments, for adult subjects, a daily dose can comprise a mass equivalent of tranexamic acid, ranging from about 100 mg to about 3,600 mg, in certain embodiments, from about 300 mg to about 3,600 mg, in certain embodiments, from about 600 mg to about 2,400 mg, and in certain embodiments, from about 600 mg to about 1,200 mg. The dose of 4-({[(2-methylpropanoyloxy)ethoxy]carbonylamino}methyl)cyclohexanecarboxylic acid and appropriate dosing intervals may be selected to maintain a sustained therapeutically effective concentration of tranexamic acid, in the blood of a subject, and in certain embodiments, without exceeding a minimum adverse concentration.

In certain embodiments, dosage forms provided by the present disclosure may be administered once per day, twice per day, and in certain embodiments at intervals of more than once per day. Dosing may be provided alone or in combination with other drugs and may continue as long as required for effective treatment of the disease. Dosing includes administering a dosage form to a mammal, such as a human, in a fed or fasted state.

A dose may be administered in a single dosage form or in multiple dosage forms. When multiple dosage forms are used the amount of 4-({[(2-methylpropanoyloxy)ethoxy]carbonylamino}methyl)cyclohexanecarboxylic acid contained within each of the multiple dosage forms may be the same or different.

Suitable daily dosage ranges for oral administration can range from about 2 mg to about 50 mg of tranexamic acid equivalents per kilogram body weight.

In certain embodiments, compound (1) may be administered to treat bleeding in a subject in an amount from about 50 mg-equivalents to about 2,000 mg-equivalents tranexamic acid per day, from about 100 mg-equivalents to about 1,500 mg-equivalents tranexamic acid per day, from about 200 mg-equivalents to about 1,000 mg-equivalents tranexamic acid per day, or in any other appropriate daily dose.

In certain embodiments, dosage forms provided by the present disclosure may be administered to treat bleeding in a subject so as to provide a therapeutically effective concentration of tranexamic acid in the blood or plasma of the subject. In certain embodiments, a therapeutically effective concentration of tranexamic acid in the blood or plasma of a subject is from about 1 μg/mL to about 60 μg/mL, from about 2 μg/mL to about 50 μg/mL, from about 5 μg/mL to about 40 μg/mL, from about 5 μg/mL to about 20 μg/mL, and in certain embodiments from about 5 μg/mL to about 10 μg/mL. In certain embodiments, a therapeutically effective concentration of tranexamic acid in the blood or plasma of a subject is at least about 2 μg/mL, at least about 5 μg/mL, at least about 10 μg/mL, at least about 15 μg/mL, at least about 25 μg/mL, and in certain embodiments at least about 30 μg/mL. In certain embodiments, a therapeutically effective concentration of tranexamic acid in the blood or plasma of a subject is less than an amount that causes unacceptable adverse effects including adverse effects to homeostasis. In certain embodiments, a therapeutically effective concentration of tranexamic acid in the blood or plasma of a subject is an amount sufficient to restore and/or maintain homeostasis in the subject.

In certain embodiments, dosage forms provided by the present disclosure may be administered to treat bleeding in a subject so as to provide a therapeutically effective concentration of tranexamic acid in the blood or plasma of a subject for an extended period of time such as, for example, for at least about 4 hours, for at least about 6 hours, for at least about 8 hours, for at least about 10 hours, and in certain embodiments, for at least about 12 hours.

The amount administered may vary depending upon whether 4-({[(2-methylpropanoyloxy)ethoxy]carbonylamino}methyl)cyclohexanecarboxylic acid is administered prophylactically prior to bleeding, during a bleeding episode, or following a bleeding episode. The amount administered may vary during a treatment regimen.

Dosage forms provided by the present disclosure may be included in a kit that may be used to administer the compound to a subject for treating bleeding. A kit may include dosage forms provided by the present disclosure suitable for administration to a subject and instructions for oral administering the dosage forms to a subject. A kit may include one or more containers for containing one or more pharmaceutical compositions and may include divided containers such as a divided bottle or a divided foil packet. A container may be any appropriate shape or form which is made of a pharmaceutically acceptable material. A particular container may depend on the dosage form and the number of dosage forms provided. Instructions provided with a kit may include directions for administration and may include a memory aid. Instructions supplied with a kit may be printed and/or supplied, for example, as an electronic-readable medium, a video cassette, an audiotape, a flash memory device, or may be published on an internet web site or distributed to a subject as an electronic mail. A memory aid may be a written memory aid, which contains information and/or instructions for the physician, pharmacist, and/or subject to facilitate compliance with a dosing regimen. A memory aid may also be mechanical or electronic. When a therapeutic regimen includes administration of 4-({[(2-methylpropanoyloxy)ethoxy]carbonylamino}methyl)cyclohexanecarboxylic acid and at least on other therapeutic agent, a kit may include the at least one other therapeutic agent in the same or separate container as 4-({[(2-methylpropanoyloxy)ethoxy]carbonylamino}methyl)cyclohexanecarboxylic acid, respectively.

Dosage forms provided by the present disclosure may further comprise one or more pharmaceutically active compounds in addition to compound (1). Such compounds may be provided to treat bleeding or to treat a disease, disorder, or condition other than bleeding.

In certain embodiments, compound (1) may be used in combination with at least one other therapeutic agent. In certain embodiments, compound (1) may be administered to a subject together with another compound for treating bleeding in the subject. In certain embodiments, the at least one other therapeutic agent may be a different tranexamic acid prodrug. Compound (1) and the at least one other therapeutic agent may act additively or, and in certain embodiments, synergistically. The at least one additional therapeutic agent may be included in the same dosage form comprising compound (1) or may be in a separate dosage form. Accordingly, methods provided by the present disclosure can further include, in addition to administering compound (1), administering one or more therapeutic agents effective for treating bleeding or a different disease, disorder or condition than bleeding. Methods provided by the present disclosure include administration of compound (1) and one or more other therapeutic agents provided that the combined administration does not inhibit the therapeutic efficacy of compound (1) and/or does not produce adverse combination effects.

In certain embodiments, dosage forms comprising compound (1) may be administered concurrently with the administration of another therapeutic agent, which may be part of the same dosage form as, or in a different dosage form than that comprising compound (1). Compound (1) may be administered prior or subsequent to administration of another therapeutic agent. In certain embodiments of combination therapy, the combination therapy may comprise alternating between administering compound (1) and a composition comprising another therapeutic agent, e.g., to minimize adverse drug effects associated with a particular drug. When compound (1) is administered concurrently with another therapeutic agent that potentially may produce an adverse drug effect including, but not limited to, toxicity, the other therapeutic agent may advantageously be administered at a dose that falls below the threshold at which the adverse drug reaction is elicited.

In certain embodiments, dosage forms comprising compound (1) may be administered with one or more substances to enhance, modulate and/or control release, bioavailability, therapeutic efficacy, therapeutic potency, stability, and the like of compound (1). For example, to enhance the therapeutic efficacy of compound (1) or its metabolite, tranexamic acid, compound (1) or a dosage form comprising compound (1) may be co-administered with one or more active agents to increase the absorption or diffusion of compound (1) or tranexamic acid from the gastrointestinal tract to the systemic circulation, or to inhibit degradation of compound (1) or tranexamic acid in the blood of a subject. In certain embodiments, a dosage form comprising compound (1) may be co-administered with an active agent having pharmacological effects that enhance the therapeutic efficacy of compound (1).

Additionally, dosage forms provided by the present disclosure may be used in combination with other drugs that are themselves known to cause bleeding.

In certain embodiments, in the treatment of a subject suffering from bleeding, such as for example menorrhagia, a dosage form comprising compound (1) may can be administered in conjunction with an agent known or believed to be effective in treating bleeding, including oral synthetic progestins such as medroxyprogesterone, norethindrone acetate, and norgestrel; natural progestins such as progesterone; gonadatrophin inhibitors such as danazol; or nonsteroidal anti-inflammatory agent such as aspirin, salsalate, diflunisal, ibuprofen, detaprofen, nabumetone, piroxicam, mefenamic acid, naproxen, diclofenac, indomethacin, sulindac, tolmetin, etodolac, ketorolac, oxaprozin, and COX-2 inhibitors such as celecoxib, meloxicam, and rofecoxib.

### EXAMPLES

The following examples describe in detail the preparation and properties of tablet dosage forms comprising compound (1). It will be apparent to those skilled in the art that many modifications, both to materials and methods, may be practiced without departing from the scope of the disclosure.

### General Experimental Protocols

trans-4-(Aminomethyl)-cyclohexanecarboxylic acid (tranexamic acid) was purchased from Sigma-Aldrich, Inc. and was used without further manipulation. O-(1-Acyloxyalkyl) S-alkylthiocarbonates were previously synthesized according to the procedures disclosed in U.S. Pat. No. 7,227,028 and converted to the corresponding acyloxyalkyl N-hydroxysuccinimide carbonic acid esters as described therein, or according to the general procedure given below. All other reagents and solvents were purchased from commercial suppliers and used without further purification or manipulation. It is understood that one of skill in the art may modify the procedures described below in order to increase or decrease the scale of each procedure.

Proton NMR spectra (400 MHz) were recorded on a Varian AS 400 NMR spectrometer equipped with an autosampler and data processing computation. DMSO-d⁶ (99.9% D) or CDCl₃ (99.8% D) were used as solvents unless otherwise noted. The DMSO or chloroform solvent signal was used for calibration of the individual spectra. Analytical LC/MS was performed on a Waters 2790 separation module equipped with a Waters Micromass QZ mass spectrometer, a Waters 996 photodiode detector, and a Merck Chromolith UM2072-027 or Phenomenex Luna C-18 analytical column. Mass-guided preparative HPLC purification of final compounds was performed on an instrument equipped with a Waters 600 controller, ZMD Micromass spectrometer, a Waters 2996 photodiode array detector, and a Waters 2700 Sample Manager. Acetonitrile/water gradients containing 0.05% formic acid were used as eluents in both analytical and preparative HPLC experiments.

### General Procedure for the Synthesis of Acyloxyalkyl N-hydroxysuccinimide Carbonic Acid Esters

A 250 mL round-bottomed flask equipped with a magnetic stir bar and a pressure-equilibrating dropping funnel was charged with the 1-acyloxyalkyl alkylthiocarbonate (about 10 mmol) and N-hydroxysuccinimide (about 20-40 mmol). Dichloromethane (about 20-40 mL) was added and the reaction mixture cooled to about 0° C. in an ice-bath. Peracetic acid (32 wt. %) in a 40-45% aqueous acetic acid solution (about 30 mmol) was added dropwise with stifling over a period of about one hour to the cooled solution. After addition was complete, stirring was continued for additional three to five hours at this temperature, the reaction being monitored by ¹H NMR spectroscopy. After complete consumption of the starting material, the reaction mixture was diluted with additional dichloromethane, and the organic solution was washed successively with water (three times) and once with a 10% aqueous solution of sodium metabisulfite or sodium thiosulfate to quench any remaining oxidant. The combined organic extracts were dried over MgSO₄, filtered, and the solvent removed under reduced pressure with a rotary evaporator. Compound identity, integrity, and purity were checked by ¹H NMR spectroscopy. The crude material was used directly in the next step, or could be further purified by commonly employed techniques well-known to those skilled in the art.

### Example 1

### 1-[(2,5-Dioxopyrrolidinyl)oxycarbonyloxy]-propyl 2-methylpropanoate (1)

Following the above general procedure, 1-(ethylthiocarbonyloxy)-propyl 2-methylpropanoate (2.3 g, 9.82 mmol) and N-hydroxysuccinimide (4.6 g, 40 mmol) were reacted in dichloromethane (20 mL) with peracetic acid (32 wt-%, 6.13 mL). After aqueous workup, isolation and removal of residual solvents in vacuo, the crude product 1 (1.76 g, 61%) was obtained as a yellow oil. The material was used in the next step without further purification. ¹H NMR (400 MHz, CDCl₃): δ=1.02 (t, J=7.6 Hz, 3H), 1.20 (d, J=6.8 Hz, 3H), 1.21 (d, J=7.2 Hz, 3H), 1.88-2.00 (m, 2H), 2.61 (hept., J=7.2 Hz, 1H), 2.84 (s, 4H), 6.71 (t, J=5.2 Hz, 1H).

### Example 2

### 1-[(2,5-Dioxopyrrolidinyl)oxycarbonyloxy]-2-methylpropyl 2-methylpropanoate

Following the above general procedure, 2-methyl-1-(methylthiocarbonyloxy)-propyl 2-methylpropanoate (2.34 g, 10.0 mmol) and N-hydroxysuccinimide (5.76 g, 50 mmol) were reacted in dichloromethane (30 mL) with peracetic acid (32 wt-%, 8.17 mL). After aqueous workup, isolation and removal of residual solvents in vacuo, the crude product 2 (2.12 g, 70%) was obtained as a pale yellow oil. The material was used in the next step without further purification. ¹H NMR (400 MHz, CDCl₃): δ=1.04 (d, J=7.2 Hz, 6H), 1.21 (d, J=6.8 Hz, 3H), 1.22 (d, J=6.8 Hz, 3H), 2.15-2.21 (m, 1H), 2.63 (hept., J=7.2 Hz, 1H), 2.84 (s, 4H), 6.59 (d, J=5.2 Hz, 1H). MS (ESI) m/z 324.10 (M+Na)⁺.

### General Nucleophilic Carbamoylation Procedure for Synthesis of Acyloxyalkyl Carbamates of Tranexamic Acid

A screw-capped 40 mL glass vial equipped with a magnetic stir bar was charged with trans-4-(aminomethyl)cyclohexanecarboxylic (tranexamic) acid (about 472 mg, about 3.0 mmol). The appropriate acyloxyalkyl N-hydroxysuccinimide carbonic acid ester (about 2.0 mmol) was added either as a solid or was dissolved in a small volume of solvent (for oily materials). A mixture of methyl tert-butyl ether (MTBE), acetone, and water (v/v/v=4:3:1) (about 15-20 mL) was added, and the reaction mixture stirred for about 12 hours at room temperature. Upon completion of the reaction, the mixture was diluted with ethyl acetate and 1 N aqueous hydrochloric acid (about 10 mL) was added. After vigorous mixing followed by phase separation, the aqueous layer was extracted once more with EtOAc, and the combined organic extracts were washed with brine. The solvents were evaporated under reduced pressure, the dry residue was dissolved in a mixture of 60% (v/v) acetonitrile/water, and the solution filtered through a 0.2 μm nylon syringe filter. Final purification was achieved by mass-guided preparative HPLC. After lyophilization of the solvents, the pure compounds were obtained as white powders.

### General Procedure for One Pot Synthesis of Acyloxyalkyl Carbamates of Tranexamic Acid

Under an atmosphere of nitrogen, a dry 100 mL round-bottomed flask equipped with a magnetic stir bar and a rubber septum was charged with trans-4-(aminomethyl)cyclohexanecarboxylic (tranexamic) acid (about 786.1 mg, about 5.0 mmol). Anhydrous dichloromethane (about 10-15 mL) was added, and the reaction mixture was cooled to about 0° C. with an ice bath. Chlorotrimethylsilane (about 1.396 mL, about 1.195 g, about 11.0 mmol) was added neat at this temperature, followed by slow addition of N-methylmorpholine (about 1.374 mL, about 1.264 g, about 12.5 mmol). The reaction mixture was stirred at this temperature for about 30 min, when an appropriately substituted chloroalkylchloroformate (about 7.5 mmol) was added dropwise and in neat form. The reaction mixture was stirred at this temperature for an additional 30 min at which time a premixed mixture of NMM (about 2.75 mL, about 2.53 g, about 25 mmol) and an appropriately substituted carboxylic acid (about 50 mmol) was added at about 0° C. The reaction mixture was stirred overnight with warming to room temperature. The dichloromethane was removed in vacuo from the dark brownish reaction mixture using a rotary evaporator. The crude reaction product was diluted with methyl tert-butyl ether (MTBE), and the solution washed three times with water. The organic layer was dried over MgSO₄, and the filtrate evaporated in vacuo using a rotary evaporator. The crude dry residue was dissolved in a small amount of a mixture of 60% (v/v) acetonitrile/water (about 5 mL), and the solution filtered through a 0.2 μm nylon syringe filter. Final purification was achieved by mass-guided preparative HPLC. After lyophilization of the solvents, the pure compounds were generally obtained as white powders.

### General Procedure for the Synthesis of Sodium Salts of Acyloxyalkyl Carbamates of Tranexamic Acid

A screw-capped 40 mL vial equipped with a magnetic stir bar was charged with an appropriately substituted acyloxyalkyl carbamate of tranexamic acid (about 5.0 mmol). The material was dissolved in about 10 mL of acetonitrile. A solution of sodium bicarbonate (NaHCO₃) (about 420.1 mg, about 5.0 mmol) in about 20 mL of water was added at room temperature and the mixture was stirred one hour after the evolution of carbon dioxide subsided. The clear solution was frozen at −78° C. and the solvents were lyophilized. After lyophilization of the solvents, the pure compounds were obtained as white powders.

### Example 3

### trans-4-{[1-(2-Methylpropanoyloxy)ethoxycarbonyl]-aminomethyl}-Cyclohexanecarboxylic Acid (3)

Following the general nucleophilic carbamoylation procedure, tranexamic acid and 1-[(2,5-dioxopyrrolidinyl)oxycarbonyloxy]ethyl 2-methylpropanoate 2 were reacted to provide the title compound 3 (333 mg, 53% yield) as a colorless powder after work-up and mass-guided preparative HPLC purification. ¹H NMR (400 MHz, DMSO-d⁶): δ=0.82-0.94 (br. m, 2H), 1.058 (d, J=6.4 Hz, 3H), 1.062 (d, J=6.8 Hz, 3H), 1.17-1.36 (br. m, 3H), 1.38 (d, J=5.6 Hz, 3H), 1.65-1.73 (br. m, 2H), 1.83-1.91 (br. m, 2H), 2.10 (tt, J=12.0, 3.6 Hz, 1H), 2.49 (hept., J=6.8 Hz, 1H), 2.77-2.85 (br. m, 2H), 6.62 (q, J=5.2 Hz, 1H), 7.45 (t, J=6.0 Hz, 1H), 11.97 (br. s, 1H). MS (ESI) m/z 338.08 (M+Na)⁺; 314.01 (M−H)⁻.

### Example 4

### Sodium trans-4-{R-(2-Methylpropanoyloxy)ethoxycarbonyl]-aminomethyl}-Cyclohexanecarboxylate (4)

Following the general procedure for the formation of the corresponding sodium carboxylates of acyloxyalkyl carbamates of tranexamic acid, 5.03 g (15.94 mmol) of trans-4-{[1-(2-methylpropanoyloxy)ethoxycarbonyl]-aminomethyl}-cyclohexanecarboxylic acid 3 was reacted with 1.34 g (15.94 mmol) of sodium bicarbonate (NaHCO₃) in 40 mL of a mixture of acetonitrile and water (1:1) to yield 5.38 g (quant.) of the title compound 4 as a colorless powder. ¹H NMR (400 MHz, DMSO-d⁶): δ=0.72-0.84 (br. m, 2H), 1.057 (d, J=6.4 Hz, 3H), 1.059 (d, J=6.8 Hz, 3H), 1.20-1.32 (br. m, 3H), 1.38 (d, J=5.2 Hz, 3H), 1.59-1.73 (br. m, 3H), 1.75-1.83 (m, 2H), 2.43-2.53 (m, 1H), 2.72-2.84 (br. m, 2H), 6.62 (q, J=5.6 Hz, 1H), 7.42 (t, J=5.6 Hz, 1H). MS (ESI) m/z 338.16 (M+Na)⁺; 314.12 (M−H)⁻.

### Example 5

### (+)-trans-4-({[(1S)-1-(2-Methylpropanoyloxy)ethoxy]carbonylamino}methyl)-Cyclohexanecarboxylic Acid (5)

The enantiomers of trans-4-{[1-(2-methylpropanoyloxy)ethoxycarbonyl]-aminomethyl}-cyclohexanecarboxylic acid 3 were resolved by means of a Waters mass-guided preparative HPLC using a ChiralPak AD-RH 250×20 mm column, an isocratic eluent of 30% acetonitrile/70% water/0.05% formic acid, and a flow rate of 15 mL/min. The enantiomeric excesses were determined with an analytical Waters 2690/ZQ LC/MS apparatus using a ChiralPak AD-RH column, an isocratic eluent consisting of 30% acetonitrile/70% water/0.05% formic acid, and a flow rate of 60 μL/min. 529 mg of the title compound 5 was obtained as a colorless powder after lyophilization [R_{f}=12.2 min; e.e.=98.3%; [α]_{D}^{25.8}=+18.64, c (19.97, MeOH)]. The assignment of the absolute configuration was accomplished by comparison with material obtained from an independent synthesis. ¹H NMR (400 MHz, DMSO-d⁶): δ=0.82-0.94 (br. m, 2H), 1.057 (d, J=6.8 Hz, 3H), 1.061 (d, J=6.8 Hz, 3H), 1.17-1.36 (br. m, 3H), 1.38 (d, J=5.6 Hz, 3H), 1.65-1.73 (br. m, 2H), 1.83-1.91 (br. m, 2H), 2.10 (tt, J=12.0, 3.6 Hz, 1H), 2.49 (hept., J=6.8 Hz, 1H), 2.77-2.85 (br. m, 2H), 6.62 (q, J=5.2 Hz, 1H), 7.45 (t, J=6.0 Hz, 1H), 11.97 (br. s, 1H). MS (ESI) m/z 338.16 (M+Na)⁺; 314.12 (M−H)⁻.

### Example 6

### Sodium trans-4-({[(1S)-1-(2-methylpropanoyloxy)ethoxy]carbonylamino}methyl)-Cyclohexanecarboxylate (6)

Following the general procedure for the formation of the corresponding sodium carboxylates of acyloxyalkyl carbamates of tranexamic acid, 90.0 mg (0.2854 mmol) of (+)-trans-4-({[(1S)-1-(2-methylpropanoyloxy)ethoxy]carbonylamino}methyl)-cyclohexanecarboxylic acid 5 was reacted with 24.0 mg (0.2854 mmol) of sodium bicarbonate (NaHCO₃) in 4 mL of a mixture of acetonitrile and water (1:1) to yield 96.3 mg (quant.) of the title compound 6 as a colorless powder. The enantiomeric excesses were determined with an analytical Waters 2690/ZQ LC/MS apparatus using a ChiralPak AD-RH column, an isocratic eluent consisting of 30% acetonitrile/70% water/0.05% formic acid, and a flow rate of 60 μL/min (R_{f}=12.1 min; e.e.=98.5%). ¹H NMR (400 MHz, DMSO-d⁶): δ=0.72-0.84 (br. m, 2H), 1.057 (d, J=6.4 Hz, 3H), 1.059 (d, J=6.8 Hz, 3H), 1.20-1.32 (br. m, 3H), 1.38 (d, J=5.2 Hz, 3H), 1.59-1.73 (br. m, 3H), 1.75-1.83 (m, 2H), 2.43-2.53 (m, 1H), 2.72-2.84 (br. m, 2H), 6.62 (q, J=5.6 Hz, 1H), 7.42 (t, J=5.6 Hz, 1H). MS (ESI) m/z 338.16 (M+Na)⁺; 314.12 (M−H)⁻.

### Example 7

### (−)-trans-4-({[(1R)-1-(2-Methylpropanoyloxy)ethoxy]carbonylamino}methyl)-Cyclohexanecarboxylic Acid (7)

The enantiomers of trans-4-{[1-(2-methylpropanoyloxy)ethoxycarbonyl]-aminomethyl}-cyclohexanecarboxylic acid 3 were resolved by means of a Waters mass-guided preparative HPLC using a ChiralPak AD-RH 250×20 mm column, an isocratic eluent of 30% acetonitrile/70% water/0.05% formic acid, and a flow rate of 15 mL/min. The enantiomeric excesses were determined with an analytical Waters 2690/ZQ LC/MS apparatus using a ChiralPak AD-RH column, an isocratic eluent consisting of 30% acetonitrile/70% water/0.05% formic acid, and a flow rate of 60 μL/min. 310 mg of the title compound 7 was obtained as a colorless powder after lyophilization [R_{f}=15.1 min; e.e.=97.6%; [α]_{D}^{25.5}=14.94, c (24.30, MeOH)]. The assignment of the absolute configuration was accomplished by comparison with material obtained from an independent synthesis. ¹H NMR (400 MHz, DMSO-d⁶): δ=0.82-0.94 (br. m, 2H), 1.057 (d, J=6.8 Hz, 3H), 1.061 (d, J=6.8 Hz, 3H), 1.17-1.36 (br. m, 3H), 1.38 (d, J=5.6 Hz, 3H), 1.65-1.73 (br. m, 2H), 1.83-1.91 (br. m, 2H), 2.10 (tt, J=12.0, 3.6 Hz, 1H), 2.49 (hept., J=6.8 Hz, 1H), 2.77-2.85 (br. m, 2H), 6.62 (q, J=5.2 Hz, 1H), 7.45 (t, J=6.0 Hz, 1H), 11.97 (br. s, 1H). MS (ESI) m/z 338.16 (M+Na)⁺; 314.12 (M−H)⁻.

### Example 8

Sodium trans-4-({[(1R)-1-(2-Methylpropanoyloxy)ethoxy]carbonylamino}methyl)-Cyclohexanecarboxylate (8)

Following the general procedure for the formation of the corresponding sodium carboxylates of acyloxyalkyl carbamates of tranexamic acid, 90.0 mg (0.2854 mmol) of (−)-trans-4-({[(1R)-1-(2-methylpropanoyloxy)ethoxy]carbonylamino}methyl)-cyclohexanecarboxylic acid 7 was reacted with 24.0 mg (0.2854 mmol) of sodium bicarbonate (NaHCO₃) in 4 mL of a mixture of acetonitrile and water (1:1) to yield 96.3 mg (quant.) of the title compound 8 as a colorless powder. The enantiomeric excesses were determined with an analytical Waters 2690/ZQ LC/MS apparatus using a ChiralPak AD-RH column, an isocratic eluent consisting of 30% acetonitrile/70% water/0.05% formic acid, and a flow rate of 60 μL/min (R_{f}=15.0 min; e.e.=97.7%). ¹H NMR (400 MHz, DMSO-d⁶): δ=0.72-0.84 (br. m, 2H), 1.057 (d, J=6.4 Hz, 3H), 1.059 (d, J=6.8 Hz, 3H), 1.20-1.32 (br. m, 3H), 1.38 (d, J=5.2 Hz, 3H), 1.59-1.73 (br. m, 3H), 1.75-1.83 (m, 2H), 2.43-2.53 (m, 1H), 2.72-2.84 (br. m, 2H), 6.62 (q, J=5.6 Hz, 1H), 7.42 (t, J=5.6 Hz, 1H). MS (ESI) m/z 338.16 (M+Na)⁺; 314.12 (M−H)⁻.

### Example 9

### Dissolution Profiles of Tablet Formulations

Dissolution profiles for tablets were obtained using a USP paddle apparatus (Type II) in 900 mL of 10 mM potassium phosphate monobasic (KH₂PO₄) buffer at pH 7.4, with 1%-vol sodium lauryl sulfate (SLS) at a temperature of 37° C. The paddle stifling speed was 50 rpm.

### Example 10

### Granulation and Tableting

Compound (1), sodium lauryl sulfate (SLS), and hydroxypropylmethyl cellulose (METHOCEL™-E4M, Dow Chemical) were weighed and sieved through an 60-mesh screen to break up soft agglomerates. The screened materials were then placed into a high shear wet granulator (Diosna P1-6, 1L bowl, 350 rpm) and pre-blended for 2 min (impeller speed of 350 rpm and chopper speed of 2000 rpm). Water was weighed out (62.5 wt-%), added to the granulator over about 45 min at 1.4 g/min by drip, and the wet mass blended for 1 min at an impeller speed of 350 rpm and a chopper speed of 2,000 rpm for both water addition and wet mass blending. Following granulation, the wet granules were spread on paper and dried at ambient conditions for 3 h, at 40° C. for 1 h and at 30° C. for 15 minutes. The dried granules were then sized by passing the granules through a Quadro Comil fitted with a 0.050G screen.

To prepare the tableting formulation, the dried granules were transferred to a V-shell, 1-quart blender and hydroxypropylmethyl cellulose (METHOCEL™ K100M, Dow Chemical), previously passed through a 35-mesh screen, was added and mixed for 10 min at 25 rpm. Magnesium stearate (NF, non-bovine, Mallinckrodt) was sieved through a 70-mesh screen, added to the blender, and the contents mixed for 3 min at 25 rpm. Tablets were prepared from the blend using a GlobePharma tableting press equipped with modified oval D tooling (0.346×0.747).

The composition of granules and tablets disclosed in this example are summarized in Table 1 and Table 2.

TABLE 1
Granule composition.

| Component | wt-% |
|---|---|
| Compound (1) | 98.0 |
| SLS | 1.0 |
| METHOCEL ™-E4M | 1.0 |

TABLE 2
Composition of tablets (wt-%).

| | Blend A | Blend B | Blend C | Blend D | Blend E |
|---|---|---|---|---|---|
| Granulation (wt-%)¹ | 88.0 | 88.0 | 92.0 | 92.0 | 92.0 |
| METHOCEL ™ | 10.0 | 10.0 | 6.0 | — | 6.0 |
| K100M (wt-%) | | | | | |
| METHOCEL ™ | — | — | — | 6.0 | — |
| K4M (wt-%) | | | | | |
| Magnesium | 2.0 | 2.0 | 2.0 | 2.0 | 2.0 |
| stearate (wt-%) | | | | | |
| Total (wt-%) | 100.0 | 100.0 | 100.0 | 100.0 | 100.0 |
| Tablet Weight (mg) | 1160 | 1160 | 1109 | 1109 | 1109 |

¹Corresponding to 98 wt-% compound (1); 1 wt-% SLS; and 1 wt-% METHOCEL ™-E4M.

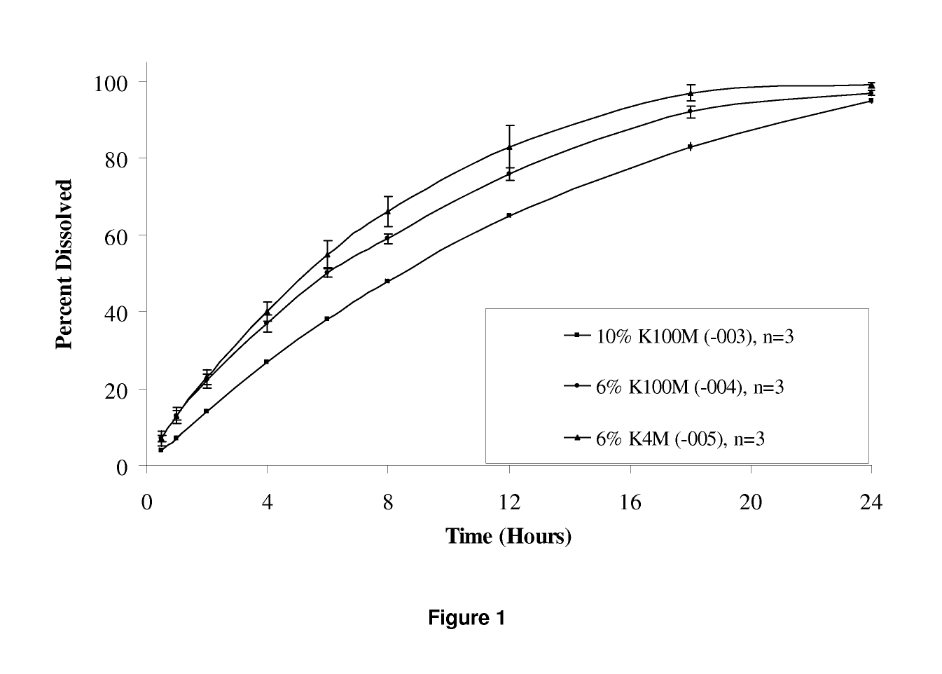

The friability of the tablets determined according to USP Friability Test No. 1216 was less than 0.3 wt-%. Dissolution profiles for tablets are shown in FIG. 1.

Finally, it should be noted that there are alternative ways of implementing the embodiments disclosed herein. Accordingly, the present embodiments are to be considered as illustrative and not restrictive. Furthermore, the claims are not to be limited to the details given herein, and are entitled their full scope and equivalents thereof.

What is claimed is:

1. A tablet dosage form comprising from about 80 wt-% to about 90 wt-% 4-({[(2-methylpropanoyloxy)ethoxy]carbonylamino}methyl)cyclohexanecarboxylic acid.

2. The tablet dosage form of claim 1, comprising from about 100 mg to about 2,000 mg 4-({[(2-methylpropanoyloxy)ethoxy]carbonylamino}methyl)cyclohexanecarboxylic acid.

3. The tablet dosage form of claim 1, comprising hydroxypropylmethyl cellulose and a lubricant.

4. The tablet dosage form of claim 3, comprising:
from about 5 wt-% to about 15 wt-% of the hydroxypropylmethyl cellulose; and
from about 1 wt-% to about 3 wt-% of the lubricant.

5. The tablet dosage form of claim 3, wherein the hydroxypropylmethyl cellulose is a hypromellose 2208 polymer having a methoxyl content from about 19% to about 24%, a hydroxypropyl content from about 7% to about 12%, and a viscosity from about 80,000 cps to about 120,000 cps in a 2% aqueous solution.

6. The tablet dosage form of claim 1, comprising granules, wherein the granules comprise the 4-({[(2-methylpropanoyloxy)ethoxy]carbonylamino}methyl)cyclohexanecarboxylic acid.

7. The tablet dosage form of claim 6, wherein the granules comprise greater than about 95 wt-% 4-({[(2-methylpropanoyloxy)ethoxy]carbonylamino}methyl)cyclohexanecarboxylic acid.

8. The tablet dosage form of claim 7, wherein the granules comprise:
a surfactant; and
a hydroxypropylmethyl cellulose polymer.

9. The tablet dosage form of claim 8, wherein the granules comprise
from about 0.5 wt-% to about 2.0 wt-% of the surfactant; and
from about 0.5 wt-% to about 2.0 wt-% of the hydroxypropylmethyl cellulose polymer.

10. The tablet dosage form of claim 8, wherein the hydroxypropyl methyl cellulose polymer of the granules is hypromellose 2910 polymer having a methoxyl content from about 28% to about 30%, a hydroxypropyl content from about 7% to about 12%, and a viscosity from about 3,000 cps to about 5,600 cps in a 2% aqueous solution.

11. The tablet dosage form of claim 8, wherein the granules consist essentially of:
about 98 wt-% 4-({[(2-methylpropanoyloxy)ethoxy]carbonylamino}methyl)cyclohexanecarboxylic acid;
about 1 wt-% of a surfactant; and
about 1 wt-% of a hypromellose 2910 polymer having a methoxyl content from about 28% to about 30%, a hydroxypropyl content from about 7% to about 12%, and a viscosity from about 3,000 cps to about 5,600 cps in a 2% aqueous solution.

12. The tablet dosage form of claim 1, wherein the dosage form is a sustained release dosage formulation.

13. The tablet dosage form of claim 1, wherein in 10 mM, pH 7.4, potassium phosphate monobasic buffer with 1% sodium lauryl sulfate at 37° C. stirred at 50 rpm (USP, Type II):
from about 20% to about 45% of the 4-({[(2-methylpropanoyloxy)ethoxy]carbonylamino}methyl)cyclohexanecarboxylic acid is released within about 4 hours;
from about 40% to about 70% of the 4-({[(2-methylpropanoyloxy)ethoxy]carbonylamino}methyl)cyclohexanecarboxylic acid is released within about 8 hours;
from about 60% to about 85% of the 4-({[(2-methylpropanoyloxy)ethoxy]carbonylamino}methyl)cyclohexanecarboxylic acid is released within about 12 hours; and
from about 80% to about 100% of the 4-({[(2-methylpropanoyloxy)ethoxy]carbonylamino}methyl)cyclohexanecarboxylic acid is released within about 20 hours.

14. A solid granulation comprising greater than about 95 wt-% 4-({[(2-methylpropanoyloxy)ethoxy]carbonylamino}methyl)cyclohexanecarboxylic acid.

15. An oral dosage form comprising the granulation of claim 14.

16. A method of treating bleeding in a subject comprising orally administering to a subject in need of such treatment at least one dosage form of any one of claim 1 and 15.

17. The method of claim 16, wherein the bleeding is menorrhagia.

18. The method of claim 16, wherein the bleeding is perioperative bleeding.

19. The method of claim 16, wherein the bleeding is caused by a wound.
